(19) United States
(12) Patent Application Publication
(10) Pub. No.: US 2007/0232575 A1
(43) Pub. Date: Oct. 4, 2007

(54) Treatment of Pulmonary Artery Hypertension with Dhea, Dheas, Dhea Analogs, or Dhea Derivatives

(76) Inventors: Etienne Emile Baulieu, Cedex (FR); Bettina Experton, Del Mar, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/587,765

(22) PCT Filed: Feb. 3, 2005
(86) PCT No.: PCT/US05/03679

§ 371 Date: Jun. 11, 2007

Related U.S. Application Data
(60) Provisional application No. 60/542,627, filed on Feb. 5, 2004.

(51) Int. Cl. A61K 31/56; A61M 15/08

(52) U.S. Cl. 514/169; 128/203.15

(57) ABSTRACT

The present invention is related to the treatment and prevention of pulmonary vascular diseases. Administration of dehydroepiandrosterone (DHEA) has been found to prevent and decrease pulmonary artery hypertension. Accordingly, the invention discloses methods of treating or preventing pulmonary vascular diseases such as pulmonary artery hypertension by pulmonary administration of compositions containing DHEA, DHEAS, DHEA analogs, or DHEA derivatives. Additionally, the DHEA, DHEAS, DHEA analogs, or DHEA derivatives may be used in combination with other pharmaceutical agents, such as bronchodilators, vasodilators, anti-inflammatory agents, and anti-infectious agents to treat pulmonary diseases.

### BACKGROUND OF THE INVENTION

1. Field of the Invention

The invention relates to the prevention/treatment of cardiovascular consequences of pulmonary alterations or diseases. In particular, the invention relates to the treatment or prevention of pulmonary artery hypertension by pulmonary administration of a pharmaceutical composition containing dehydroepiandrosterone.

2. Description of the Related Art

Pulmonary artery hypertension (“PAH”) occurs when the blood pressure in the arteries of the lungs is abnormally high. This often occurs when the arterioles within the lung become narrowed. The arterial narrowing creates resistance and an increased work load for the heart, in particular eventually causing the right ventricle of the heart to become enlarged and weakened. Without treatment, the disease often develops into congestive heart failure.

Pulmonary artery hypertension may occur when no other heart or lung diseases or alterations are causing the increase in blood pressure (termed “primary artery pulmonary hypertension”). Alternatively, secondary pulmonary artery hypertension may occur when other underlying diseases are causing the high blood pressure in the pulmonary arterioles. Examples of underlying diseases that can result in a clinical diagnosis of pulmonary artery hypertension include, for example, breathing disorders such as emphysema or bronchitis. Smoking may also result in pulmonary artery hypertension. Other diseases such as HIV or lupus may also result in a diagnosis of pulmonary artery hypertension. Children may also be susceptible to pulmonary artery hypertension. Pulmonary artery hypertension may also occur during hypoxia due to the effects of high altitude. Other causes include incidents during anesthesia and cardiac or pulmonary surgery. Additionally, genetic abnormalities can be the cause of some forms of pulmonary artery hypertension. Other commonly used terms for pulmonary artery hypertension include pulmonary hypertension, sporadic primary pulmonary hypertension, and familial primary pulmonary hypertension. Symptoms of pulmonary artery hypertension may include, for example, shortness of breath or light-headedness during activity, chest pain, fatigue, fainting, dizziness, and leg swelling. Currently, primary PAH is often treated using inhaled nitric oxide or i.v. or s.c. enoprostenol (prostacyclin) (Vachiery, J. L., et al. (2002) Chest 121, 1561-1565, which is incorporated by reference herein in its entirety).

Dehydroepiandrosterone (DHEA) is a naturally occurring steroidal androgen precursor that is synthesized by the adrenal gland, and is largely secreted and circulating in blood in form of DHEA sulfate (DHEAS). The secretion and blood levels of DHEAS have been found to decrease dramatically upon aging. In elderly individuals, there is an inverse correlation between the natural level of DHEA present and the presence of functional limitations, such as confinement, dyspnea, depression, or self-perception of poor health (Berr et al. (1996), Proc. Natl. Acad. Sci. USA 93:13410-13415; Mazat et al. (2001), Proc. Natl. Acad. Sci. USA 98:8145-8150). Exogenous application of DHEA has been administered for many uses, including the “treatment” of aging. DHEA administration to elderly individuals has also resulted in increased skin hydration, increased libido, and increased bone turnover (Baulieu et al., (2000) Proc. Natl. Acad. Sci. USA 97:4279-4284, which is incorporated by reference herein in its entirety).

### SUMMARY OF THE INVENTION

In some embodiments of the present invention, a method for reducing pulmonary arterial pressure (PAP) is provided, by introducing an effective amount of a DHEA, DHEAS, DHEA analog, or DHEA derivative into the pulmonary airways of a mammal. In some aspects, the DHEA, DHEAS, DHEA analog, or DHEA derivative has the general formula

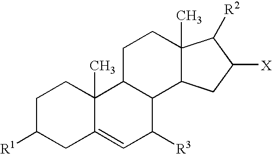

wherein

X is H or halogen; R¹, R² and R³ are independently ═O, —OH, —SH, H, halogen, pharmaceutically acceptable ester, pharmaceutically acceptable thioester, pharmaceutically acceptable ether, pharmaceutically acceptable thioether, pharmaceutically acceptable inorganic esters, spirooxirane, spirothirane, —OSO₂ R⁵ or —OPOR⁵R⁶, or a pharmaceutically acceptable monosaccharide, disaccharide or oligosaccharide; R⁵ and R⁶ are independently —OH, pharmaceutically acceptable esters or pharmaceutically acceptable ethers; and pharmaceutically acceptable salts.

In some aspects, the introduction is by inhalation, inspiration, or nebulization. In additional aspects, the effective amount of DHEA, DHEAS, DHEA analog, or DHEA derivative is from about 0.01 mg per kg body weight to about 100 mg per kg body weight per day. The introduction of DHEA, DHEAS, DHEA analog, or DHEA derivative can be by means of chronic administration or intermittent administration.

In yet additional aspects of the invention, the DHEA, DHEAS, DHEA analog, or DHEA derivative can be in the form of a dry particulate or an aerosol. The effective amount of DHEA, DHEAS, DHEA analog, or DHEA derivative can be, for example, from about 0.01 mg per kg body weight to about 100 mg per kg body weight per day. Further aspects of the invention include the additional administration of antimicrobial agents, such as an antibacterial agent, an antifungal agent, or an antiviral agent. Yet additional aspects include the additional administration of a vasodilator or a bronchodilator or a steroidal or non-steroidal anti-inflammatory drug.

In another embodiment of the invention, a metered dose inhaler having at least one compound selected from the group consisting of: DHEA, DHEAS, a DHEA analog, or a DHEA derivative is provided. In some aspects of the invention, the metered dose inhaler may contain additional compounds, such as an antibacterial agent, antifungal agent, an antiviral agent, a vasodilator, or a bronchodilator or a steroidal or non-steroidal anti-inflammatory drug.

In yet another embodiment of the invention, a dry powder inhaler having at least one compound selected from the following group: DHEA, DHEAS, a DHEA analog, or a DHEA derivative. In some aspects, the compound formulation has a particle size of about 0.5 μm to about 5 μm.

In another embodiment of the invention, a method of treatment of pulmonary hypertension is provided, wherein at least one of the following compounds is administered: DHEA, DHEAS, a DHEA analog, or a DHEA derivative. In some aspects, administration may be by injection, or by oral means. In additional aspects, the administration is pulmonary administration, such as by use of an aerosol. In further aspects, the method is used to treat pulmonary hypertension that is caused by disorders of the respiratory system. In other aspects, the method is used to treat pulmonary hypertension that is caused by chronic hypoxia. In additional aspects, the method is used to treat or chronic hypoxic pulmonary hypertension.

In an additional embodiment of the invention, a method of reversing the severity of pulmonary hypertension is provided by administering at least one of the following compounds: DHEA, DHEAS, a DHEA analog, or a DHEA derivative.

In a further embodiment of the invention, a method for decreasing RV wall thickness is provided, by administering at least one of the following compounds: DHEA, DHEAS, a DHEA analog, or a DHEA derivative.

In yet further embodiments of the invention, methods for preventing or decreasing intrapulmonary artery remodeling (IPA) and prevention of increased pulmonary arterial pressure are provided, by administering at least one of the following compounds: DHEA, DHEAS, a DHEA analog, or a DHEA derivative. Additional aspects include the reduction of [Ca²⁺]i in pulmonary artery smooth muscle cells (PASMCs) by oral, injected or pulmonary administration of DHEA, DHEAS, a DHEA analog, or a DHEA derivative. Additional aspects of the invention include the prevention or reduction of cardiac right ventricle hypertrophy by oral, injected or pulmonary administration of DHEA, DHEAS, a DHEA analog, or a DHEA derivative. Further aspects of the invention include the activation of BKCa and Kv channels by the oral, injected or pulmonary administration of DHEA, DHEAS, a DHEA analog, or a DHEA derivative. Yet further aspects of the invention include the increased expression of BKCa, or reduction in downregulation of BKCa, by the oral, injected or pulmonary administration of DHEA, DHEAS, a DHEA analog, or a DHEA derivative.

### BRIEF DESCRIPTION OF THE DRAWINGS

FIG. 1 is a bar graph demonstrating the effects of oral DHEA on pulmonary and systemic circulation. Oral administration of DHEA for 3 weeks decreased the pulmonary arterial pressure (PAP) (A) and right ventricular (RV) wall thickness (B) but did not affect systemic circulation (C) of chronic hypoxic/DHEA-treated (CH-DHEA) rats or cardiac function (D and E).

FIG. 2 is a bar graph demonstrating the effects of intravascular DHEA on mean PAH. Intravascular DHEA had no effect in both control and CH-DHEA-treated animals (A and C), whereas it induced a significant decrease in PAP in CH animals (B).

FIG. 3 is a graph showing the effect of intravascular DHEA on mean PAP. Intravascular DHEA (3 μg to 3 mg/kg) decreased the PAH of CH rats in a dose-dependent manner.

FIG. 4 is a bar graph showing the effects of oral DHEA on PA remodeling. CH induced a significant increase in the PA wall thickness. Oral administration of DHEA for 3 weeks or 1 week prevented and reversed PA wall remodeling, respectively.

FIG. 5 is a bar graph showing effect of oral DHEA on [Ca²⁺]i of pulmonary artery smooth muscle cells (PASMCs). Oral administration of DHEA for 3 weeks or 1 week induced a significant decrease in [Ca²⁺]i of PASMCs.

FIG. 6 is a bar graph demonstrating the effect of in vitro DHEA administration on K channels of PASMCs. (A) DHEA (100 μM) induced a significant decrease in [Ca²⁺]i. IbTx (100 nM) blocked 65% of the DHEA-induced [Ca²⁺]i decrease, and combined IbTx and 4-AP (1 mM) was 100% efficient. Agitoxin-2 had no effect on the DHEA response. (B) DTT suppressed the DHEA-induced decrease in [Ca²⁺]i. The 1H-[1,2,4]oxadiazolol[4,3,-a]quinoxalin-1-one (ODQ), genistein, and a PKA inhibitor had no effect on DHEA-induced [Ca²⁺]i.

FIG. 7 is a line graph showing the effects of DHEA on PA reactivity to BKCa blockers and BKCa expression. The graph demonstrates in vitro concentration—response curves for the effect of IbTx on the resting tension of IPA rings from CH and CH-DHEA rats. The amplitude of contraction is expressed as a percentage of the KCl (80 mM)-induced response obtained at the beginning of the experiments. Note the increase in the IbTx response in rings from CH-DHEA rats. Data points are mean±SEM for CH (n=11, N=4) and CH-DHEA (n=11, N=4) rats.

### DETAILED DESCRIPTION OF THE PREFERRED EMBODIMENT

The present invention relates to the finding that dehydroepiandrosterone (DHEA), DHEA sulfate (DHEAS), DHEA analogs, or DHEA derivatives can be beneficial in the treatment of pulmonary vascular diseases. In some embodiments of the invention, pulmonary administration of DHEA, DHEAS, DHEA analogs, or DHEA derivatives can be used to treat pulmonary artery hypertension in mammals. Furthermore, the activity of DHEA on the vascular system appears to be specific for pulmonary circulation.

DHEA can be safely administered to individuals in need of treatment of pulmonary artery hypertension. DHEA has been used in short- and long-term human studies without major toxicity (Baulieu, E. E., et al. (2000) Proc. Natl. Acad. Sci. USA 97, 4279-4284, which is incorporated by reference herein in its entirety), and even large amounts of DHEA have been safely administered to human patients (Tummala, S. & Svec, F. (1999) Clin. Biochem. 32, 355-361, which is incorporated by reference herein in its entirety).

The pulmonary administration of DHEA, DHEAS, DHEA analogs, or DHEA derivatives may be used to treat pulmonary artery hypertension and related diseases. As used herein, the term “treat” or “treatment” refer to both therapeutic treatment and prophylactic or preventative measures, wherein the object is to prevent or slow down (lessen) an undesired physiological change or disorder. The term “treat” also refers to the characterization of the type or severity of disease which may have ramifications for future prognosis, or need for specific treatments. For purposes of this invention, beneficial or desired clinical results include, but are not limited to, lessening or prevention of pulmonary hypertension, alleviation of symptoms, decreased PAH, decreased PAP, decreased right ventricular (RV) wall thickness, diminishment of extent of pulmonary artery hypertension, stabilized (i.e., not worsening) state of pulmonary artery hypertension, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

In humans, the presence of severe pulmonary disease is frequently associated with chronic hypoxia (CH). Human PAH is defined by a resting pulmonary arterial pressure (PAP) of >20 mmHg (Weitzenblum, E. (2003) Heart 89, 225-230, which is incorporated by reference herein in its entirety). PAH secondary to disorders of the respiratory system is the most frequent cause of PAH. Chronic hypoxia is the principal pathophysiological mechanism of this PAH.

Exposure of animals to hypoxia leads to the development of chronic hypoxic-pulmonary artery hypertension (CH-PAH). Therefore, a rat-based model of PAH was developed in order to further understand PAH mechanisms and to examine possible treatment methods. Accordingly, chronic hypoxia was induced in rats using a hypobaric chamber at 0.5 atm that corresponds to a stable hypoxia with an inspired fraction of oxygen of 10%, as detailed in Example 2. Animals were typically placed in the chamber for 7-21 days. This model induces PAH similar to human PAH secondary to disorders of respiratory system, such as chronic obstructive pulmonary disease, interstitial lung disease, or neonatal chronic obstructive lung disease secondary of prematurity.

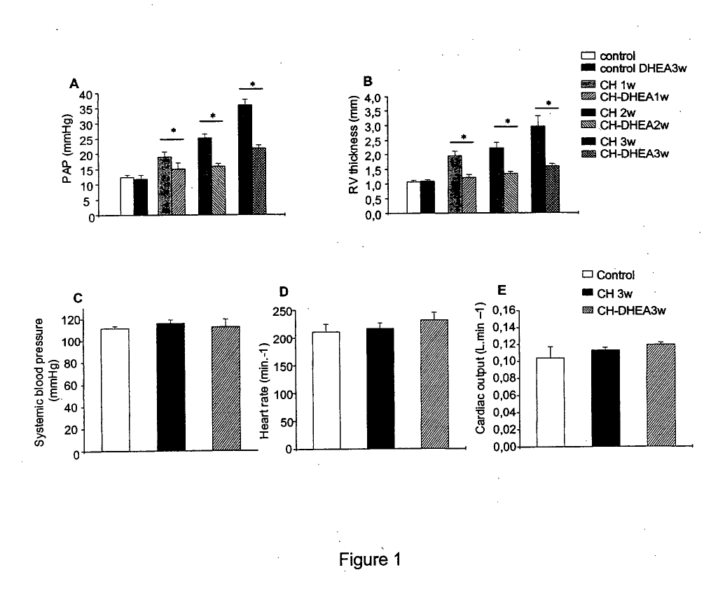

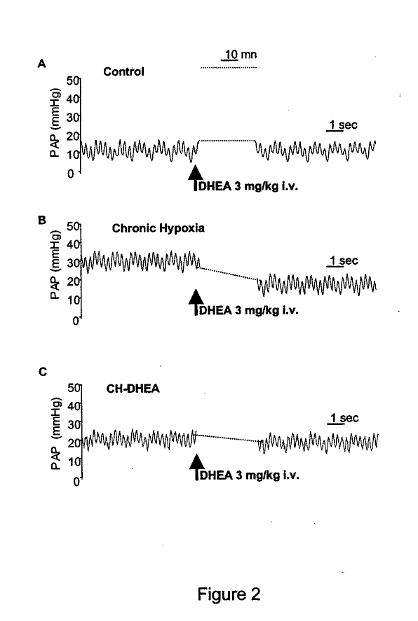

This chronic hypoxic-pulmonary artery hypertension model was used to study the effects of DHEA treatment on pulmonary artery (PA) hypertension (Examples 6 and 7). Oral administration of DHEA to rats at approximately 30 mg/kg every alternate day was found to almost entirely prevent increases in PA pressure, cardiac right ventricle hypertrophy, and PA remodeling (FIG. 1). Furthermore, a single intravascular dose of DHEA at 3 mg/kg, or a 1-wk orally administered DHEA regimen (30 mg/kg every alternate day) in hypertensive rats significantly decreased PAH (FIG. 2).

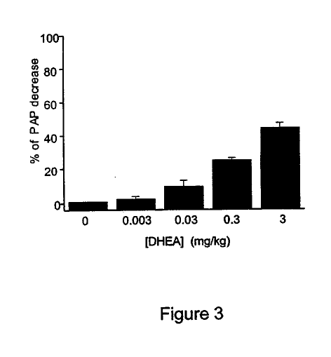

Further, FIG. 3 shows that intravascular administration of DHEA to CH-PAH rats can act in a dose-dependent manner. While a 0.03 mg/kg treatment allowed about a 10% decrease of PAP, a ten-fold increase in dosage to 0.3 mg/kg resulted in an approximate 25% decrease in PAP, and another ten-fold increase in DHEA to 3.0 mg/kg resulted in an approximate 45% decrease of PAH.

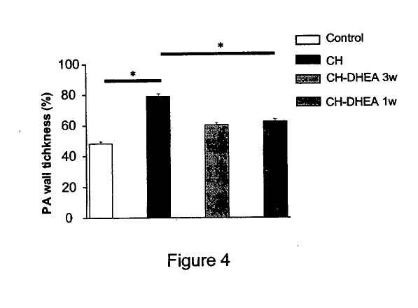

CH-PAH involves pulmonary arterial vasoconstriction and remodeling. To determine the degree of changes in the intrapulmonary artery (IPA) wall upon CH treatment, isometric contractions were measured in rings from IPAs as described in Example 9. The results (FIG. 4) show that while the CH-PAH rats exhibited an increase in PA wall thickness when compared to normoxic control rats, oral DHEA administration to CH-PAH rats prevented the PA wall remodeling. Additionally, oral DHEA treatment for 1 week decreased the PA remodeling.

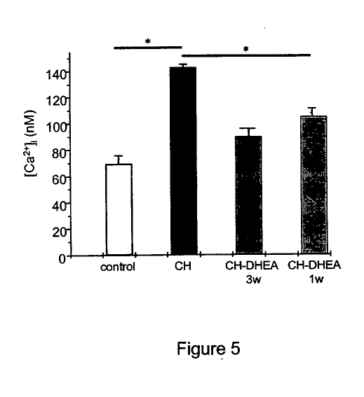

Pulmonary hypertension involves the action of both endothelium and vascular smooth muscle cells (SMCs) (Michelakis, E. D. & Weir, E. K. (2001) Clin. Chest Med. 22, 419-432, which is incorporated by reference herein in its entirety). Both the contractile status and the proliferative status of SMCs are regulated by the levels of intracellular Ca²⁺ ([Ca²⁺]i). The [Ca²⁺]i levels are determined in part by the influx of Ca²⁺ through the voltage-gated, L-type Ca²⁺ channels. Therefore, to determine whether chronic hypoxia and DHEA treatments are capable of altering intracellular calcium levels, [Ca²⁺]i levels were measured using microspectrofluorimetry (Example 10). The chronic hypoxia-treated (CH) rats had a significant increase in [Ca²⁺]i in vascular smooth muscle cells (SMCs) as compared to normoxic rats) (FIG. 5). However, oral administration of DHEA to the CH rats for 3 weeks or 1 week resulted in a decrease in [Ca²⁺]i as compared to the CH-PAH rats.

In pulmonary artery (PA) SMCs, the membrane potential is regulated by large conductance Ca²⁺-activated channels (BKCa) (Peng, W., et al. (1997) Am. J. Physiol. 272, C1271-C1278) and voltage-gated K+ channels (Kv), including shaker family Kv (Archer, S. L., et al. (2000) Adv. Exp. Med. Biol. 475, 219-240; Patel, A. J., et al. (1997) EMBO J. 16, 6615-6625, all of which are incorporated by reference herein in their entireties). K channel (BKCa and Kv) function and expression are down-regulated with development and maintenance of CH-PAH (Platoshyn, O., et al. (2001) Am. J. Physiol. 280, L801-L812; Olschewski, A., et al. (2002) Am. J. Physiol. 283, L1103-L1109, which are incorporated by reference herein in their entireties). CH reduces K current density in PASMCs, resulting in a state of depolarization (Reeve, H. L., et al. (2001) J. Appl. Physiol. 90, 2249-2256; Smirnov, S. V., et al. (1994) Am. J. Physiol. 266, H365-H370, which are incorporated by reference herein in their entireties), followed by elevation of [Ca²⁺]i, which induces contraction and proliferation (Platoshyn, O., et al. (2000) Am. J. Physiol. 279, C1540-C1549, which is incorporated by reference herein in its entirety). The mechanism for K channel down-regulation suggests that it may be related to the altered redox state induced by CH, and lungs of rats with CH-PAH are in a more reduced redox state than those of normoxic controls, as indicated by increased levels of reduced glutathione (Reeve, H. L., et al. (2001) supra). A reduced redox state has potential for both short-term effects through modulation of K+ channels function (Reeve, H. L., et al. (1995) Exp. Physiol. 80, 825-834, which is incorporated by reference herein in its entirety) and long-term effects by activating several oxygen-responsive genes including hypoxia-inducible factor (HIF) (Huang, L. E., et al. (1996) J. Biol. Chem. 271, 32253-32259, which is incorporated by reference herein in its entirety).

The effect of DHEA appears to involve a large conductance Ca²⁺-activated potassium channel (BKCa)-dependent stimulatory mechanism. DHEA is a BKCa opener in hypoxic human pulmonary cells (Peng, W., et al. (1999) Am. J. Respir. Cell Mol. Biol. 20, 737-745, which is incorporated by reference herein in its entirety), which can shift the redox balance toward an oxidized state leading to both BKCa and Kv activation and thus repolarization of the PASMCs membrane potential and its effects on the [Ca²⁺]i.

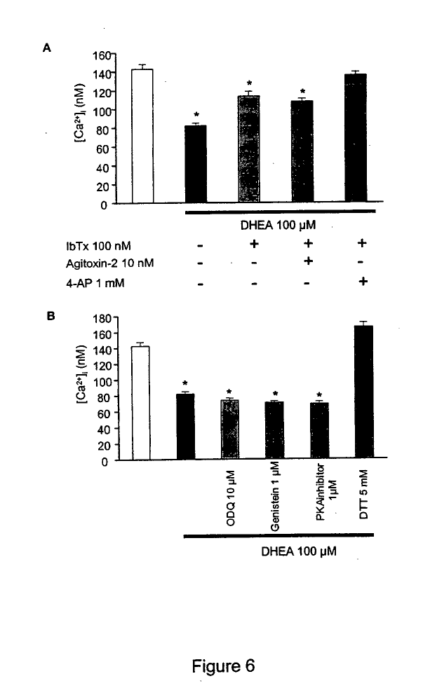

To investigate the effect of DHEA on the potassium channels BKCa and Kv, PASMCs from CH rats were treated with both DHEA (100 μM) and IbTx (100 nM) for 10 minutes, as described in Example 11. This treatment partially inhibited the change induced by DHEA alone on the resting [Ca²⁺]i value by 62% (n=30; N=4) (FIG. 6A). The addition of agitoxin-2 had no effect (n=15; N=4). Combined presence of 4-AP and iberiotoxin (IbTx) almost totally abolished the DHEA effect on the resting [Ca²⁺]i (n=20; N=4).

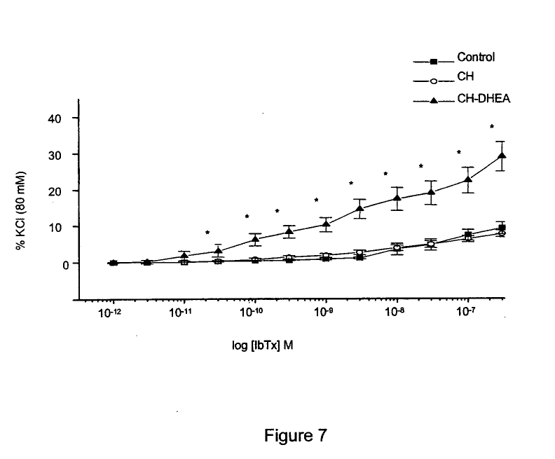

Chronic hypoxia had no significant effects on the IbTx induced contraction of IPA rings from CH rats compared with control rats (see Example 11). However, the administration of DHEA was found to increase the sensitivity for IbTx in IPA rings (FIG. 7). Further, an immunoblot analysis of BKCa levels in arterial pulmonary extracts determined that the BKCa protein was significantly decreased in the CH group vs. the control group. This decrease was prevented by the 3 week DHEA treatment. Taken together, it appears that the effect of DHEA treatment on the function and expression of BKCa is the main factor explaining the ability of the compound to prevent and reverse CH-PAH, and that the DHEA effects involve a redox-dependent pathway.

Results from cellular studies indicates that PAH is associated with an increase in [Ca²⁺]i secondary to membrane depolarization of the PA myocytes (Yuan, X. J. (1995) Circ. Res. 77, 370-378, which is incorporated by reference herein in its entirety). This leads to a deep change in the PA properties, and PA from CH rats do not respond to further acute hypoxic stress. IPA rings from CH-DHEA animal develop higher reactivity to IbTx, which blocks KCa as compared with CH rats, suggesting an involvement of the BKCa in the control of IPA basal tone from CH-DHEA rats (FIG. 7). Furthermore, in smooth muscle cells from CH-PAH rats, acute exposure to DHEA induced a decrease of the resting [Ca²⁺]i that is blocked by IbTx. Western blot analysis indicates an up-regulation of the -subunit BKCa in CH-DHEA as compared with CH rats.

Under hypoxic conditions, the redox balance of the PA cells is in a reduced state, as described (Reeve, H. L., et al. (2001) J. Appl. Physiol. 90, 2249-2256; Yuan, X. J., et al. (1995) Exp. Physiol. 80, 803-813). This reduced state is responsible in part for the K channel inhibition leading to CH-PAH. It has been suggested that DHEA may induce a decrease in the NADH:NAD ratio leading to oxidation of the cells (Gupte, S. A., et al. (2002) J. Pharmacol. Exp. Ther. 301, 299-305, which is incorporated by reference herein in its entirety). In view of this data, a possible cellular mechanism, by which DHEA activates BKCa, may be correlated to a cellular oxidation leading to potassium channel activation. The fact that the reducing agent DTT inhibits the effect of DHEA on [Ca²⁺]i (FIG. 6) suggests that DHEA activated both BKCa and Kv by changing the redox balance toward a more oxidative state (Gupte, S. A., et al. (2002) J. Pharmacol. Exp. Ther. 301, 299-305; Swierczynski, J., et al. (2001) Pol. J. Pharmacol. 53, 125-130).

Further, the results described herein are of a pharmacological nature. The free plus conjugated DHEA concentration in the rat plasma is <1 nM. Very little DHEA is synthesized in rat adrenals and gonads. The active doses of DHEA used in the experiments herein, for chronic oral as well as acute intravascular administrations, induce circulating concentration of the order of 10⁻⁷ M of DHEA(S), as also are DHEA concentrations used in in vitro studies.

DHEA is metabolized to form a number of compounds in rats (and all animals) including testosterone, estradiol, and 5-androstene-3β,17β-diol, and others. The i.v. administration of testosterone, estradiol, and pregnenolone, following the same intravascular protocol in CH-DHEA rats for each compound (3 mg/kg) as for DHEA, did not reveal any effect of these steroids.

In contrast, 5-androsten-3β,17β-diol,3β-hydroxy-5α-androstan-17-one, and 3βmethyl-5-androsten-17-one did reverse PA hypertension. Whether the steroids directly interact with KCa and/or Kv has not yet been demonstrated at the molecular level. In humans, the circulating concentration of DHEA(S) is of the order of 10⁻⁶ to 10⁻⁵ M, and the very majoritarian physiological sulfate form may have the potential to create efficacious levels of DHEA with reference to these rat experiments and effects recorded on chronically hypoxic human pulmonary smooth-muscle levels (Peng, W., et al. (1999) Am. J. Respir. Cell Mol. Biol. 20, 737-745, which is incorporated by reference herein in its entirety).

DHEA can be Used to Treat Pulmonary Hypertension

In some embodiments of the invention, the DHEA composition is comprised of DHEA. In other embodiments, the DHEA composition contains the DHEA sulfate (DHEAS) form of DHEA. In yet other embodiments, DHEA derivatives and/or DHEA analogs are used. Further embodiments include compositions that are mixtures containing more than one form of DHEA, DHEAS, DHEA analog, or DHEA derivative.

The timing of administration of the compositions of the invention can depend on several factors, such as the severity of the disease, the health of the patient, the presence of additional diseases, the response of the patient to the treatment, and the presence of other compounds in the composition of the invention.

“Acute” administration as used herein refers to administration of the DHEA, DHEAS, DHEA analog, or DHEA derivative compositions over a short period of time, to deliver a therapeutically effective amount of the composition of the invention in a small number of dose administrations, such as, for example, a single dose.

“Chronic” administration as used herein refers to administration of the DHEA, DHEAS, DHEA analog, or DHEA derivative compositions of the present invention in a continuous mode as opposed to an acute mode, so as to maintain the desired effect for an extended period of time.

“Intermittent” administration is treatment that is not consecutively done without interruption, but rather is periodic in nature.

Administration “in combination with” one or more further therapeutic agents includes simultaneous (concurrent) and consecutive administration in any order.

An “individual” is a vertebrate, preferably a mammal, more preferably a human.

“Mammal” for purposes of treatment refers to any animal classified as a mammal, including humans, domestic and farm animals, and zoo, sports, or pet animals, such as dogs, horses, cats, cows, etc. Preferably, the mammal herein is human.

The DHEA, DHEAS, DHEA analog, or DHEA derivative compositions of the present invention may be administered by several routes. In preferred embodiments of the present invention, the route of administration is pulmonary administration, for example, by way of inhalation or inspiration. In some embodiments of the present invention, the route of administration is by oral means. In other embodiments of the present invention, administration of the DHEA, DHEAS, DHEA analog, or DHEA derivative compositions of the present invention occurs by injection, introduction or infusion by intravenous, intraperitoneal, intracerebral, subcutaneous, epicutaneous, intranasal, intratracheal, intrapulmonary, nebulized, intramuscular, intraocular, intraarterial, intracerebrospinal, or intralesional routes, or by sustained release systems as noted below. In yet other embodiments of the invention, the DHEA, DHEAS, DHEA analog, or DHEA derivative compositions of the present invention may also be administered transdermally, for example, by use of an ointment, lotion, or skin patch.

DHEA may be present in the free form, or may be present as DHEA sulfate (DHEAS) other salts, or as a mixture. DHEA analogs or derivatives and or their salts are also contemplated for use in the invention. In some embodiments, the side groups of the DHEA, DHEAS, DHEA analog, or DHEA derivative may include, for example, esters, thioesters, ethers, thioethers, sugar residues, and the like. Examples of DHEA analogs or derivatives that may be useful for the method of the present invention can be found, for example, in U.S. Pat. No. 5,753,640, which is incorporated by reference herein in its entirety.

DHEA analogs or derivatives can include, for example, compounds having the formula

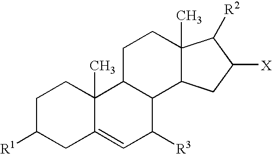

wherein

X is H or halogen;

R¹, R² and R³ are independently ═O, —OH, —SH, H, halogen, pharmaceutically acceptable ester, pharmaceutically acceptable thioester, pharmaceutically acceptable ether, pharmaceutically acceptable thioether, pharmaceutically acceptable inorganic esters, spirooxirane, spirothirane, —OSO₂ R⁵ or —OPOR⁵R⁶, or a pharmaceutically acceptable monosaccharide, disaccharide or oligosaccharide;

R⁵ and R⁶ are independently —OH, pharmaceutically acceptable esters or pharmaceutically acceptable ethers; and

pharmaceutically acceptable salts.

In general, the DHEA analogs or derivatives may include but are not limited to limited to the following:

R² is ═O, R³ and X are each H and R¹ is ═O, —OH, pharmaceutically acceptable esters thereof, pharmaceutically acceptable ethers thereof or pharmaceutically acceptable salts;

R² is ═O, R³ is H, X is halogen and R¹ is ═O, —OH, pharmaceutically acceptable esters thereof, pharmaceutically acceptable ethers thereof or pharmaceutically acceptable salts;

R² is ═O, R³ and X are each H and R¹ is —SH, pharmaceutically acceptable thioesters thereof, pharmaceutically acceptable thioethers thereof or pharmaceutically acceptable salts;

R² is ═O, R³ is H, X is halogen and R¹ is —SH, pharmaceutically acceptable thioesters thereof, pharmaceutically acceptable thioethers thereof or pharmaceutically acceptable salts;

R² is ═O, X is H and R¹ and R³ are independently ═O, —OH, pharmaceutically acceptable esters thereof, pharmaceutically acceptable ethers thereof or pharmaceutically acceptable salts;

R² is ═O, X is halogen and R¹ and R³ are independently ═O, —OH, pharmaceutically acceptable esters thereof, pharmaceutically acceptable ethers thereof or pharmaceutically acceptable salts;

R² is ═O, X is H and R¹ and R³ are independently —SH, pharmaceutically acceptable thioesters thereof, pharmaceutically acceptable thioethers thereof or pharmaceutically acceptable salts;

R² is ═O, X is halogen and R¹ and R³ are independently —SH, pharmaceutically acceptable thioesters thereof, pharmaceutically acceptable thioethers thereof or pharmaceutically acceptable salts;

R² is —OH, R³ and X are each H and R¹ is ═O, —OH, pharmaceutically acceptable esters thereof, pharmaceutically acceptable ethers thereof or pharmaceutically acceptable salts;

R² is —OH, R³ is H, X is halogen and R¹ is ═O, —OH, pharmaceutically acceptable esters thereof, pharmaceutically acceptable ethers thereof or pharmaceutically acceptable salts;

R² is —OH, R³ and X are each H and R¹ is —SH, pharmaceutically acceptable thioesters thereof, pharmaceutically acceptable thioethers thereof or pharmaceutically acceptable salts;

R² is —OH, R³ is H, X is halogen and R¹ is —SH, pharmaceutically acceptable thioesters thereof, pharmaceutically acceptable thioethers thereof or pharmaceutically acceptable salts;

R² is —OH, X is H and R¹ and R³ are independently ═O, —OH, pharmaceutically acceptable esters thereof, pharmaceutically acceptable ethers thereof or pharmaceutically acceptable salts;

R² is —OH, X is halogen and R¹ and R³ are independently ═O, —OH, pharmaceutically acceptable esters thereof, pharmaceutically acceptable ethers thereof or pharmaceutically acceptable salts;

R² is —OH, X is H and R¹ and R³ are independently —SH, pharmaceutically acceptable thioesters thereof, pharmaceutically acceptable thioethers thereof or pharmaceutically acceptable salts;

R² is —OH, X is halogen and R¹ and R³ are independently —SH, pharmaceutically acceptable thioesters thereof, pharmaceutically acceptable thioethers thereof or pharmaceutically acceptable salts;

R² is —SH, R³ and X are each H and R¹ is ═O, —OH, pharmaceutically acceptable esters thereof, pharmaceutically acceptable ethers thereof or pharmaceutically acceptable salts;

R² is —SH, R³ is H, X is halogen and R¹ is ═O, —OH, pharmaceutically acceptable esters thereof, pharmaceutically acceptable ethers thereof or pharmaceutically acceptable salts;

R² is —SH, R³ and X are each H and R¹ is —SH, pharmaceutically acceptable thioesters thereof, pharmaceutically acceptable thioethers thereof or pharmaceutically acceptable salts;

R² is —SH, R³ is H, X is halogen and R¹ is —SH, pharmaceutically acceptable thioesters thereof, pharmaceutically acceptable thioethers thereof or pharmaceutically acceptable salts;

R² is —SH, X is H and R¹ and R³ are independently ═O, —OH, pharmaceutically acceptable esters thereof, pharmaceutically acceptable ethers thereof or pharmaceutically acceptable salts;

R² is —SH, X is halogen and R¹ and R³ are independently ═O, —OH, pharmaceutically acceptable esters thereof, pharmaceutically acceptable ethers thereof or pharmaceutically acceptable salts;

R² is —SH, X is H and R¹ and R³ are independently —SH, pharmaceutically acceptable thioesters thereof, pharmaceutically acceptable thioethers thereof or pharmaceutically acceptable salts;

R² is —SH, X is halogen and R¹ and R³ are independently —SH, pharmaceutically acceptable thioesters thereof, pharmaceutically acceptable thioethers thereof or pharmaceutically acceptable salts;

X is H and R¹, R² and R³ are independently ═O, —OH, a sugar residue, pharmaceutically acceptable esters thereof, pharmaceutically acceptable ethers thereof or pharmaceutically acceptable salts, wherein at least one of R¹, R² and R³ is a sugar residue;

X is halogen and R¹, R² and R³ are independently ═O, —OH, a sugar residue, pharmaceutically acceptable esters thereof, pharmaceutically acceptable ethers thereof or pharmaceutically acceptable salts, wherein at least one of R¹, R² and R³ is a sugar residue;

X is H and R¹, R² and R³ are independently ═O, —OH, pharmaceutically acceptable inorganic esters thereof or pharmaceutically acceptable salts, wherein at least one of R¹, R² and R³ is an inorganic ester;

X is halogen and R¹, R² and R³ are independently ═O, —OH, pharmaceutically acceptable inorganic esters thereof or pharmaceutically acceptable salts, wherein at least one of R¹, R² and R³ is an inorganic ester.

The R² group may also be a methyl group, or may be a longer aliphatic hydrocarbon chain (or chains), preferably of 2-14 carbon atoms, which is saturated, or partly or fully dehydrogenated. The R² group may also have additional chemical substituents.

Similarly, The R³ group may also be a methyl group, or may be a longer aliphatic hydrocarbon chain (or chains), preferably of 2-14 carbon atoms, which is saturated, or partly or fully dehydrogenated. The R³ group may also have additional chemical substituents.

In some embodiments, the substituents in the R¹, R² and/or R³ positions may be in the α or β position. In additional embodiments, 5α and/or 5β isomers may be present.

Examples of pharmaceutically acceptable esters or thioesters include, but are not limited to, esters or thioesters of the formula —OOCR or —SOCR, wherein R is a pharmaceutically acceptable alkyl, alkenyl, aryl, alkylaryl, arylalkyl, sphingosine or substituted sphingolipid groups, such as propionate, enanthate, cypionate, succinate, decanoate and phenylpropionate esters, and the like.

Suitable pharmaceutically acceptable inorganic esters include, but are not limited to, inorganic esters of the formula —OSO₂R⁵ or —OPOR⁵R⁶, wherein R⁵ and R⁶ are independently —OH, pharmaceutically acceptable esters, pharmaceutically acceptable ethers, and the like.

Examples of pharmaceutically acceptable ethers or thioethers include, but are not limited to, ethers or thioethers of the formula —OR or —SR, wherein R is as defined above or enol, or —OR⁴ is an unsubstituted or substituted spirooxirane or —SR is a spirothirane, and the like.

Examples of suitable sugar residues include, but are not limited to monosaccharides, disaccharides and oligosaccharides, and the like.

Inhalation Administration

We have discovered that one particularly advantageous mode of administration for the DHEA, DHEAS, DHEA analog, or DHEA derivative compositions of the present invention is through direct pulmonary administration. Examples 13-15, 18, and 20-24 demonstrate the pulmonary administration of a DHEA formulation by inhalation. As used herein, the term “pulmonary administration” refers to administration of a formulation of the invention through the lungs by inhalation or infusion. In specific examples, intake can occur by self-administration of a formulation of the invention while inhaling, or by administration via a respirator, e.g., to a patient on a respirator. The term “inhalation” used with respect to a formulation of the invention is synonymous with “pulmonary administration.”

In some embodiments, the present invention contemplates formulations comprising DHEA, DHEAS, DHEA analog, or DHEA derivative compositions for use in a wide variety of devices that are designed for the delivery of pharmaceutical compositions and therapeutic formulations to the respiratory tract. In some embodiments, the route of administration is in the aerosol or inhaled form. In some embodiments of the present invention, liquid or dry aerosol formulations of DHEA, DHEAS, DHEA analog, or DHEA derivative compositions of the present invention are preferably aerosolized by dispersion in a flowing air or other physiologically acceptable gas stream in a conventional manner.

As used herein, the term “aerosol” refers to a suspension in the air. In particular, aerosol refers to particle formation and its suspension in the air. According to the present invention, an aerosol formulation is a formulation comprising DHEA, DHEAS, DHEA analog, or DHEA derivative compositions of the present invention that is suitable for aerosolization, i.e., particle formation and suspension in the air, for inhalation or pulmonary administration. The DHEA, DHEAS, DHEA analog, or DHEA derivative compositions of the present invention, optionally combined with a solubilizing or dispersing agent, or dispersant, can be administered in an aerosol formulation as a dry powder or in a solution or suspension with a diluent.

With either the liquid or dry powder aerosol formulation, the formulation can be administered as an aerosol particle. The term “aerosol particle” is used herein to describe the liquid or solid particle suitable for pulmonary administration, i.e., that will reach the alveoli, and then pass through tissue to reach pulmonary artery branches.

In general, the mass median dynamic diameter will preferably be 5 micrometers or less in order to ensure that the drug particles reach the lung alveoli (Wearley, L. L., 1991, 1991, Crit. Rev. in Ther. Drug Carrier Systems 8:333, which is incorporated by reference herein in its entirety). Other considerations such as construction of the delivery device, additional components in the formulation and particle characteristics are important. These aspects of pulmonary administration of a drug are well known in the art, and manipulation of formulations, aerosolization means and construction of a delivery device require at most routine experimentation by one of ordinary skill in the art.

With regard to construction of the delivery device, any form of aerosolization known in the art, including but not limited to nebulization, atomization or pump aerosolization of a liquid formulation, and aerosolization of a dry powder formulation, can be used in the practice of the invention. Systems of aerosol delivery, such as the pressurized metered dose inhaler and the dry powder inhaler are disclosed in Newman, S. P., Aerosols and the Lung, Clarke, S. W. and Davia, D. editors, pp. 197-22 and can be used in connection with the present invention. Often, the aerosolization of a liquid or a dry powder formulation will require a propellant. The propellant may be any propellant generally used in the art. Specific nonlimiting examples of such useful propellants are a chlorofluorocarbon, a hydrofluorocarbon, a hydrochlorofluorocarbon, or a hydrocarbon, including trifluoromethane, dichlorodifluoromethane, dichlorotetrafluoroethanol, and 1,1,1,2-tetrafluoroethane, or combinations thereof.

The liquid aerosol formulations can be used with a nebulizer. Commercially available nebulizers for liquid formulations, including jet nebulizers and ultrasonic nebulizers are useful for administration. Liquid formulations can be directly nebulized, and lyophilized powder can be nebulized after reconstitution. Alternatively, these compounds can be aerosolized using a fluorocarbon formulation and a metered dose inhaler, or inhaled as a lyophilized and milled powder. Any nebulizer known in the art can be used in conjunction with the present invention such as but not limited to: Ultravent, Mallinckrodt, Inc. (St. Louis, Mo.); the Acorn II nebulizer (Marquest Medical Products, Engelwood Colo.). Examples of other nebulizers useful in conjunction with the present invention are described in U.S. Pat. Nos. 4,624,251; 3,703,173; 3,561,444; and 4,635,627. Illustrations of the use of a nebulizer for administration of DHEA, DHEAS, DHEA analog, or DHEA derivative compositions of the invention can be found in Examples 13, 15, 22, 24, and 26.

In some embodiments of the invention, the device for aerosolization is a metered dose inhaler. A metered dose inhaler provides a specific dosage when administered, rather than a variable dose depending on administration. Such a metered dose inhaler can be used with either a liquid or a dry powder aerosol formulation. Metered dose inhalers are well known in the art. Illustrations of the use of a metered dose inhaler for administration of DHEA, DHEAS, DHEA analog, or DHEA derivative compositions of the invention can be found in Examples 14, 18, 20, 21, 23, and 25.

In some embodiments of the present invention, the DHEA, DHEAS, DHEA analog, or DHEA derivative compositions of the present invention can be used as a dry powder inhaler formulation comprising a finely divided, dry powder form of the DHEA and a dispersant. Solid forms of DHEA, DHEAS, a DHEA analog, or a DHEA derivative can be obtained through standard techniques. In another embodiment, the dry powder formulation will comprise a finely divided dry powder containing DHEA, DHEAS, a DHEA analog, or a DHEA derivative a dispersing agent and also a bulking agent. Bulking agents useful in conjunction with the present formulation include such agents as lactose, sorbitol, sucrose, or mannitol, in amounts that facilitate the dispersal of the powder from the device. Dry powder inhalers and metered dose inhalers and related technologies may be obtained, for example, from Nektar Therapeutics, (San Carlos, Calif.). Nanoparticle technologies are also useful in forming suitable dry power formulations, and particle sizes as small as 100 nm or less can be used.

As used herein, the term “dispersant” or “dispersing agent” refers to an agent that assists aerosolization of DHEA, DHEAS, a DHEA analog, or a DHEA derivative in lung tissue, or both. Preferably the dispersant is pharmaceutically acceptable. Suitable dispersing agents are well known in the art, and include but are not limited to surfactants and the like. Nonlimiting examples of such surfactants are surfactants such as polyoxyethylene fatty acid esters and alcohols, and polyoxyethylene sorbitan fatty acid esters. Amounts of surfactants used will vary, being generally within the range or 0.001 and 4% by weight of the formulation. Suitable surfactants are well known in the art, and can be selected on the basis of desired properties, depending on the specific formulation, concentration of DHEA, diluent (in a liquid formulation) or form of powder (in a dry powder formulation), etc.

In some embodiments of the present invention, the aerosol formulation may include, as optional ingredients, pharmaceutically acceptable carriers, diluents, solubilizing or emulsifying agents, surfactants and excipients. Such carriers may serve simply as bulking agents when it is desired to reduce the DHEA, DHEAS, DHEA analog, or DHEA derivative concentration in the powder or liquid which is being delivered to a patient, but may also serve to enhance the stability of the composition and to improve the dispersability of the powder or liquid within a dispersion device in order to provide more efficient and reproducible delivery of the DHEA, DHEAS, DHEA analog, or DHEA derivative to improve handling characteristics of the such as flowability and consistency to facilitate manufacturing and powder or liquid filling. Other advantageous carriers include aerodynamically light particles made of a biodegradable material and having a tap density of less than 0.4 g/cm³ and a mass mean diameter between 5 and 30 μm. Examples of such particles are presented in Hanes, et al, U.S. Pat. No. 6,136,295.

Once the DHEA, DHEAS, DHEA analog, or DHEA derivative composition reaches the lung, a number of formulation-dependent factors effect the drug absorption. It will be appreciated that in treating pulmonary hypertension, such factors as aerosol particle size, aerosol particle shape, particle solubility, the presence or absence of infection, lung disease or emboli may affect the absorption of DHEA.

For each of the formulations described herein, certain lubricators, absorption enhancers, stabilizers or suspending agents may be appropriate. The choice of these additional agents will vary depending on the goal.

As an example of aerosol administration, the DHEA composition is introduced into the subject in the aerosol form in an amount between about 0.01, 0.05, or 0.08 mg of DHEA, DHEAS, DHEA analog, or DHEA derivative per kg of body weight to about 30, 50, 75, or 100 mg of DHEA, DHEAS, DHEA analog, or DHEA derivative per kg of body weight. Preferably, the dosage is in a range of about 0.1, 0.2, 0.5 or 1 mg of DHEA, DHEAS, DHEA analog, or DHEA derivative per kg of body weight to about 3.0, 5.0, 10, or 20 mg of DHEA, DHEAS, DHEA analog, or DHEA derivative per kg of body weight. In a specific embodiment, the dosage is dosage per day. One of ordinary skill in the art can readily determine a volume or weight of aerosol corresponding to this dosage based on the concentration of DHEA, DHEAS, DHEA analog, or DHEA derivative in an aerosol formulation of the invention; alternatively, one can prepare an aerosol formulation which with the appropriate dosage of DHEA, DHEAS, DHEA analog, or DHEA derivative in the volume to be administered, as is readily appreciated by one of ordinary skill in the art. Because aerosol delivery is localized rather than systemic, the effective dose will generally be lower than is required for systemic administration.

In a further embodiment, an aerosol formulation of the present invention can include other active ingredients in addition to the DHEA, DHEAS, DHEA analog, or DHEA derivative. In a preferred embodiment, such active ingredients are those used for the treatment of pulmonary artery hypertension.

Parenteral Administration

The DHEA, DHEAS, DHEA analog, or DHEA derivative composition can be formulated for parenteral administration by injection, e.g., by bolus injection or continuous infusion. Example 16 demonstrates the pulmonary administration of a DHEA formulation by twice-weekly injections. As used herein, the term “parenteral” refers to introduction of a DHEA into the body by other than the intestines, and in particular, intravenous (i.v.), intraarterial (i.a.), intraperitoneal (i.p.), intramuscular (i.m.), intraventricular, and subcutaneous (s.c.) routes. DHEA formulations for injection can be presented in unit dosage form, e.g., in ampoules or in multi-dose containers. The compositions can take such forms as suspensions, solutions or emulsions in oily or aqueous vehicles, and can contain formulatory agents such as suspending, stabilizing or dispersing agents. Alternatively, the active ingredient can be in powder form for constitution with a suitable vehicle, e.g., sterile pyrogen-free water, before use.

In addition to the formulations described previously, the DHEA, DHEAS, DHEA analog, or DHEA derivative compositions can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

Sustained Release Preparations

The DHEA, DHEAS, DHEA analog, or DHEA derivative compositions can also be formulated as a sustained-release formulation. Suitable examples of sustained-release preparations include semipermeable polymer matrices in the form of shaped articles, e.g. films, or microcapsules. Sustained release matrices include polyesters, hydrogels, polylactides (U.S. Pat. No. 3,773,919, EP 58,481), copolymers of L-glutamic acid and gamma ethyl-L-glutamate (Sidman et al., Biopolymers, 22: 547-556 (1983)), poly(2-hydroxyethyl-methacrylate) (Langer et al., J. Biomed. Mater. Res., 15: 167-277 (1981) and Langer, Chem. Tech., 12: 98-105 (1982)), ethylene vinyl acetate (Langer et al., supra) or poly-D-(−)-3-hydroxybutyric acid (EP 133,988).

In some embodiments of the invention, sustained-release compounds may include liposomally entrapped DHEA, DHEAS, DHEA analog, or DHEA derivative compositions. A liposome formulation may be particularly effective for administration of DHEA, particularly when long term administration is desired (See Wearley, 1991, Crit. Rev. in Ther. Drug Carrier Systems 8: 333). Liposomes containing compound are prepared by methods known per se: DE 3,218,121; Epstein et al., Proc. Natl. Acad. Sci. USA, 82: 3688-3692 (1985); Hwang et al., Proc. Natl. Acad. Sci. USA, 77: 4030-4034 (1980); EP 52,322; EP 36,676; EP 88,046; EP 143,949; EP 142,641; Japanese patent application 83-118008; U.S. Pat. Nos. 4,485,045 and 4,544,545; and EP 102,324. Ordinarily the liposomes are of the small (about 200-800 Angstroms) unilamelar type in which the lipid content is greater than about 30 mol. % cholesterol, the selected proportion being adjusted for the optimal therapy.

Oral Administration

In some embodiments, the DHEA, DHEAS, DHEA analog, or DHEA derivative compositions can be administered using an oral route. Example 17 demonstrates the oral administration of a DHEA formulation. For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate. talc or silica); disintegrants (e.g., potato starch or sodium starch glycolate); or wetting agents (e.g., sodium lauryl sulfate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g. methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound. For buccal administration the compositions can take the form of tablets or lozenges formulated in conventional manner.

Formulations and Dosages

The DHEA, DHEAS, DHEA analog, or DHEA derivative composition will be formulated, dosed, and administered in a fashion consistent with good medical practice. Factors for consideration in this context include the level of pulmonary hypertension being treated, the clinical condition of the individual patient, the site of delivery of the compound, the particular type of compound, the method of administration, the scheduling of administration, and other factors known to medical practitioners.

The amount to be administered will depend, for example, upon the therapeutic objectives, the route of administration, the type of compound employed, and the condition of the patient. The “therapeutically effective amount” of such a compound to be administered will be governed by such considerations, and is the minimum amount necessary to prevent, ameliorate, or treat pulmonary hypertension. Such amount is preferably below the amount that is toxic to the host or renders the host significantly more susceptible to infections.

The DHEA, DHEAS, DHEA analog, or DHEA derivative composition can be administered in various dosage quantities and schedules. The dosage can generally be in a range of about 0.01, 0.05, or 0.08 mg of DHEA, DHEAS, DHEA analog, or DHEA derivative per kg of body weight to about 30, 50, 75, or 100 mg of DHEA, DHEAS, DHEA analog, or DHEA derivative per kg of body weight. Preferably, the dosage is in a range of about 0.1, 0.2, or 0.4 mg of DHEA, DHEAS, DHEA analog, or DHEA derivative per kg of body weight to about 5.0, 10, or 20 mg of DHEA, DHEAS, DHEA analog, or DHEA derivative per kg of body weight. More preferably, a dosage is from about 0.5, 1.0, or 1.5 mg of DHEA, DHEAS, DHEA analog, or DHEA derivative per kg of body weight to about 2.0, 2.5, or 3.0 mg of DHEA, DHEAS, DHEA analog, or DHEA derivative per kg of body weight.

As noted above, however, these suggested amounts of compound are subject to a great deal of therapeutic discretion, including the individual type of compound being used. The key factor in selecting an appropriate dose and scheduling is the result obtained, as indicated above. For example, the DHEA, DHEAS, DHEA analog, or DHEA derivative composition may be optionally formulated with one or more agents currently used to prevent or treat pulmonary hypertension. The effective amount of such other agents depends on the amount of the compound present in the formulation, the clinical level of pulmonary hypertension, and other factors discussed above. These are generally used in the same dosages and with administration routes as used hereinbefore or about from 1 to 99% of the heretofore employed dosages.

The choice of schedule for DHEA, DHEAS, DHEA analog, or DHEA derivative dosage administration can depend on several factors, including but not limited to the mode of administration, the degree of severity of the pulmonary hypertension, the overall health of the patient, and the choice of formulation. The dosage may be administered, for example, as a continuous dosage, or several times a day, once a day, or more rarely such as once a week. Alternatively, the DHEA, DHEAS, DHEA analog, or DHEA derivative composition may be administered on a one-time basis, for example to a patient experiencing an acute pulmonary hypertension event. The DHEA, DHEAS, DHEA analog, or DHEA derivative formulations of the invention may include additional pharmaceutically acceptable ingredients. As used herein, the term “pharmaceutically acceptable” means approved by a regulatory agency of the Federal or a state government as listed in the U.S. Pharmacopeia or other generally recognized pharmacopeia for use in animals, and more particularly in humans.

Therapeutic formulations of DHEA, DHEAS, DHEA analog, or DHEA derivative compositions are prepared for storage by mixing DHEA, DHEAS, DHEA analog, or DHEA derivative compositions having the desired degree of purity with optional physiologically acceptable carriers, excipients, or stabilizers (Remington: The Science and Practice of Pharmacy, 19th Edition, Alfonso, R., ed, Mack Publishing Co. (Easton, Pa.: 1995)).

The DHEA, DHEAS, DHEA analog, or DHEA derivative formulation may include a carrier. The carrier is a macromolecule which is soluble in the circulatory system and which is physiologically acceptable where physiological acceptance means that those of skill in the art would accept injection of said carrier into a patient as part of a therapeutic regime. The carrier preferably is relatively stable in the circulatory system with an acceptable plasma half life for clearance. Such carrier materials may be combined with the DHEA, DHEAS, DHEA analog, or DHEA derivative prior to administration, i.e., by adding the carrier material to the buffer solution. In that way, the carrier material will be formed simultaneously with and as part of the DHEA particles. Alternatively, the carriers may be separately prepared in a dry powder or liquid form and combined with the DHEA, DHEAS, DHEA analog, or DHEA derivative by blending. The size of the carrier particles may be selected to improve the flowability of the powder or liquid, typically being in the range from 25 μm to 100 μm.

Acceptable carriers, excipients or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

In preferred embodiments, the DHEA, DHEAS, DHEA analog, or DHEA derivative composition to be used for in vivo administration is sterile. This is readily accomplished by filtration through sterile filtration membranes, prior to or following lyophilization and reconstitution. The DHEA, DHEAS, DHEA analog, or DHEA derivative composition can be stored in various forms, including but not limited to a dry powder, lyophilized cake, aqueous solution, oil-based solution, and the like. In some embodiments, injectable forms of the DHEA, DHEAS, DHEA analog, or DHEA derivative can be placed into a container having a sterile access port, for example, an intravenous solution bag or vial having a stopper pierceable by a hypodermic injection needle.

In some embodiments, the dosage forms of DHEA, DHEAS, DHEA analog, or DHEA derivative for use in treating subjects suffering from a pulmonary artery hypertension may contain a pharmaceutically acceptable diluent. Pharmaceutically acceptable diluents include but are not limited to sterile water, saline, buffered saline, dextrose solution, and the like. In a specific embodiment, a diluent that may be used in the present invention or the pharmaceutical formulation of the present invention is phosphate buffered saline, or a buffered saline solution generally between the pH 7.0-8.0 range, or water.

The pH of the DHEA, DHEAS, DHEA analog, or DHEA derivative composition will generally be in the range of between about 4.5, 5.0, 5.5, or 6.0 to about 8.0, 8.3, or 8.5. Preferably, the pH will be in the range of between about 6.5, 6.8, 7.0, or 7.2 to about 7.3, 7.5, or 7.8.

Pulmonary Therapy with DHEA, DHEAS DHEA Analogs, or DHEA Derivatives

The DHEA, DHEAS, DHEA analog, or DHEA derivative of the invention is useful in the prophylactic or therapeutic treatment of pulmonary artery hypertension in which pulmonary administration is desirable or in which the lungs are involved. The invention contemplates pulmonary administration of such amounts of DHEA, DHEAS, DHEA analog, or DHEA derivative that are sufficient either to achieve systemic delivery of a therapeutic or biological amount of DHEA, DHEAS, DHEA analog, or DHEA derivative, or such amounts that achieve only local delivery of a therapeutic or biological amount of DHEA, DHEAS, DHEA analog, or DHEA derivative to the lung. The invention further contemplates parenteral administration or pulmonary administration of DHEA, DHEAS, DHEA analog, or DHEA derivative for the treatment of pulmonary artery hypertension. It will be appreciated by one skilled in the art that goal of systemic or local delivery will depend on the indication being treated.

What constitutes a therapeutically effective amount in a particular case will depend on a variety of factors within the knowledge of the skilled practitioner. Such factors include the physical condition of the subject being treated, the severity of the condition being treated, the disorder or disease being treated, and so forth. In general, any statistically significant attenuation of one or more symptoms associated with a pulmonary artery hypertension constitutes treatment within the scope of the present invention.

It is contemplated that DHEA, DHEAS, DHEA analog, or DHEA derivative or more preferably the formulations of the present invention, can be administered to a subject in need of prophylactic or therapeutic treatment. As used herein, the term “subject” refers to an animal, more preferably a mammal, and most preferably a human.

Pulmonary administration of DHEA, DHEAS, DHEA analog, or DHEA derivative can be used to result in systemic or local effects. In another embodiment of the present invention the DHEA, DHEAS, DHEA analog, or DHEA derivative is delivered via the airways to treat diseases or disorders. As pointed out above, pulmonary administration of DHEA, DHEAS, a DHEA analog, or a DHEA derivative is preferred for the treatment of lung disorders or diseases because of the high local concentration of DHEA, DHEAS, DHEA analog, or DHEA derivative that can be delivered, the localization of significant amounts of the DHEA in extravascular space, and the ability to limit or minimize systemic effects of the DHEA, DHEAS, DHEA analog, or DHEA derivative.

Administration of DHEA, DHEAS, DHEA Analogs or DHEA Derivatives in Combination with Other Compounds

DHEA, DHEAS, DHEA analogs or DHEA derivatives may be useful in combination with other pharmaceutically useful compositions. Examples of suitable bronchodilators, vasodilators, anti-infectious agents, and other compounds which may be useful in combination with administration of one or more DHEA, DHEAS, DHEA analog, or DHEA derivatives can be found, for example, in U.S. Pat. Nos. 6,286,507, 6,257,232, 6,638,534, all of which are incorporated herein by reference in their entireties.

For example, the DHEA, DHEAS, DHEA analog, or DHEA derivative may be combined with suitable bronchodilators. Examples of bronchodilators that may be used include but are not limited to albuterol, salmeterol, formoterol, theophylline, aminophylline, disodium cromoglycate, procaterol hydrochloride, trimetoquinol hydrochloride, diprophylline, clorprenaline hydrochloride, orciprenaline sulfate, pirbuterol, hexoprenaline sulfate, bitolterol mesylate, clenbuterol hydrochloride, terbutaline sulfate, fenoterol hydrobromide, methoxyphenamine hydrochloride, and the like. An illustration of the use of the combination of DHEA and the bronchodilator albuterol is shown in Example 26.

Further, the DHEA, DHEAS, DHEA analog, or DHEA derivative may be combined with suitable vasodilators. Examples of vasodilators that may be used include but are not limited to nifedipine, isosorbide dinitrate, diltiazem hydrochloride, verapamil, nicardipine, and the like. A demonstration of the use of the combination of DHEA and the vasodilator nifedipine is shown in Example 25.

The DHEA, DHEAS, DHEA analog, or DHEA derivative of the invention may also be suitable combined with anti-infectious agents. For example, the DHEA, DHEAS, DHEA analog, or DHEA derivative may be combined with antibiotics. Antibiotics that may be useful in combination with the DHEA, DHEAS, DHEA analog, or DHEA derivative of the invention include but are not limited to Amoxicillin, Ampicillin, Benzylpenicillin, Bacampicillin, Carbenicillin, Mezlocillin, Piperacillin, Ticarcillin, Cloxacillin, Dicloxacillin, Methicillin, Oxacillin, Penicillin G (Benzathine, Potassium, Procaine) Penicillin V, Nafcillin, Cephalosporin, Cefadroxil, Cefazolin, Cephalexin, Cephalothin, Cephapirin, Cephradine, Cefaclor, Cefamandol, Cefonicid, Cefotetan, Cefoxitin, Cefprozil, Ceftmetazole, Cefuroxime, Cefuroxime, axetil, Loracarbef, Cefdinir, Ceftibuten, Cefoperazone, Cefepime, Azithromycin, Clarithromycin, Clindamycin, Dirithromycin, Erythromycin, Lincomycin, Troleandomycin, Cinoxacin, Ciprofloxacin, Enoxacin, Gatifloxacin, Levofloxacin, Lomefloxacin, Moxifloxacin, Nalidixic, acid, Norfloxacin, Ofloxacin, Sparfloxacin, Trovafloxacin, Aztreonam, Amikacin, Gentamicin, Kanamycin, Neomycin, Netilmicin, Streptomycin, Tobramycin, Paromomycin, Teicoplanin, Vancomycin, Demeclocycline, Doxycycline, Methacycline, Minocycline, Oxytetracycline, Tetracycline, Chlortetracycline, Mafenide, Sulfadiazine, Sulfacetamide, Sulfadiazine, Sulfamethoxazole, Sulfasalazine, Sulfisoxazole, Trimethoprim-Sulfamethoxazole, Rifabutin, Rifampin, Rifapentine, Linezolid, Quinopristin+Dalfopristin, Bacitracin, Chloramphenicol, Fosfomycin, Isoniazid, Methenamine, Metronidazol, Mupirocin, Nitrofurantoin, Nitrofurazone, Novobiocin, Polymyxin, Spectinomycin, Trimethoprim, Colistin, Cycloserine, Capreomycin, Ethionamide, Pyrazinamide, and the like. Examples of the use of the combination of DHEA and an antibacterial agent are shown in Examples 21 and 22.

The DHEA, DHEAS, DHEA analog, or DHEA derivative may be combined with anti-viral agents. Examples of anti-viral agents which may be useful include but are not limited to acyclovir, ganciclovir, and the like. An illustration of the use of the combination of DHEA and the anti-viral agent acyclovir is shown in Example 24.

The DHEA, DHEAS, DHEA analog, or DHEA derivative may be combined with anti-fungal agents. An illustration of the use of the combination of DHEA and an anti-fungal agent is shown in Example 23. Suitable anti-fungal agents that may be used include but are not limited to azole, nystatin, ketoconazole, clotrimazole, fluconazole, diflucan, and the like. Other compounds which may be useful in combination with administration of one or more DHEA, DHEAS, DHEA analog, or DHEA derivatives can be found in U.S. Pat. Nos. 6,286,507, 6,257,232, 6,638,534, all of which are incorporated herein by reference in their entireties.

The DHEA, DHEAS, DHEA analog, or DHEA derivative may also be combined with steroidal or non-steroidal anti-inflammatory agents. Examples of suitable non-steroidal anti-inflammatory agents that may be used include but are not limited to aspirin, diclofenac, meclofenamate, mefenamic acid, meloxicam, nabumetone, naproxen, oxaprozin, phenylbutazone, piroxicam, sulindac, tenoxicam, diflunisal, tiaprofenic acid, tolmetin, etodolac, fenoprofen, floctafenine, flurbiprofen, ibuprofen, indomethacin, ketoprofen, and the like.

Examples of steroidal anti-inflammatory agents that may be used include but are not limited to fluticasone, flunisolide, budesonide, prednisone, prednisolone, methylprednisolone, hydrocortisone, clobetasol, halobetasol, triamcinolone, betamethasone, fluocinolone, and the like.

In some embodiments of the invention, DHEA, DHEAS, DHEA analog, or DHEA derivative is administered at the same time, in the same dosage as the additional pharmaceutical agent. For example, both compounds can be combined in one metered dose inhaler. In additional embodiments of the invention, the two pharmaceutical agents are administered by different means (such as inhalation of DHEA and oral administration of the second pharmaceutical agent). In some embodiments, the two pharmaceutical agents may be administered at the same time, following the same schedule, or may be administered with a different schedule. Additionally, the DHEA, DHEAS, DHEA analog, or DHEA derivative can be administered in combination with more than one other pharmaceutical agent. For example, in some situations, the DHEAS, DHEA analog, or DHEA derivative may be administered along with both a bronchodilator and an antibacterial agent. The choice of pharmaceutical combination employed may depend on many factors, including, for example, the presence of other types of pulmonary disease, the severity of the disease, the likelihood of the development of a pulmonary viral, bacterial, or fungal infection, the age and health of the patient, and other factors.

The above disclosure generally describes the present invention. A more complete understanding can be obtained by reference to the following specific examples which are provided herein for purposes of illustration only and are not intended to limit the scope of the invention.

### EXAMPLES

Source of Chemicals used in the experiments listed herein: Collagenase (type CLS1) was obtained from Worthington Biochemical Corp. (Lakewood, N.J.). Pronase (type E), elastase (type 3), BSA, iberiotoxin (IbTx) (BKCa inhibitor), DHEA (prasterone), 1H-[1,2,4]oxadiazolol[4,3,-a]quinoxalin-1-one (ODQ) (GMPc pathway inhibitor), Genistein (tyrosine kinase inhibitor), “476485” a PKA inhibitor, 4-amino-pyridine (4-AP), DTT (a reducing agent), and agitoxin-2 (K shaker family blocker) were from Sigma Chemical Company (St. Louis, Mo.). DHEA, Indo-1, and (476485) were dissolved in either DMSO or in ethanol. The maximal concentration of DMSO and ethanol used in these experiments was <0.1% and had no effect on the mechanical activity of rings and the resting value of the [Ca²⁺]i.

Statistical analyses for the experiments described herein were performed by using NCSS 5.0 software (NCSS, Kaysville, Utah). Values were expressed as mean±SEM. Contractions were expressed as a percentage of K⁺-rich (80 mM) solution-induced contractions. Intergroup differences were assessed by a repeated-measures ANOVA or factorial ANOVA, as appropriate. Post hoc analysis used a Fisher multiple comparison test. For some comparisons, the Pearson product moment correlation coefficient between pairs of variables was used as indicated (ORIGIN 5.0, OriginLab, Northampton, Mass.). Regarding the number of experiments, (n) refers to the number of rings or cells and (N) to the number of animals. Differences were considered significant when P<0.05.

### Example 1

### Tissue and Cell Preparations and Measurements

The heart and lungs were removed and intrapulmonary arteries (IPAs) (150-300 μm of internal diameter) were then dissected. The adventitial and intimal layers were removed. For contraction experiments, rings (3 mm in length) were prepared. PASMCs were isolated by using an enzymatic protocol described in Bonnet, S., et al. (2002, Cardiovasc. Res. 53, 1019-1028, which is incorporated by reference herein in its entirety).

Lung sections were formalin-fixed in preparation for histology studies. PA external diameter (PAED), PA internal diameter (PAID), and percentage vessel wall thickness (PAED−PAID)/PAED×100) were measured in small- and medium-sized PAs (80-150 μm). Each group was comprised of four rats, and 10 measures were made per rat by an investigator blinded to the treatment groups.

### Example 2

### Chronic Hypoxia and DHEA Treatments in Rats

To study the mechanisms of PAH and its possible treatments, an animal model was utilized. The placement of rats in a hypobaric chamber for 7 to 21 days induced chronic hypoxia, resulting in CH-PAH, a useful model system for studying mammalian PAH.

The rat model experiments were performed as follows. Adult male Wistar rats (220-240 g) were randomized into five groups. Two groups were housed at normal atmospheric pressure (101 kPa); one group comprised rats treated with DHEA (30 mg/kg orally every alternate day), which induces a circulating DHEA sulfate level of 0.2 μM after 3 wk (normoxic DHEA group) and another group did not receive DHEA (normoxic group). The rat groups receiving the CH treatment were kept in a hypobaric chamber (0.5 atm; 1 atm=101.3 kPa) for 7-21 days: one group received DHEA (CH-DHEA group), another did not receive DHEA (CH group) and, in a third group, DHEA was given to the CH rats from day 15 to day 21 to look for CH-PAH regression. Intravascular administration was performed via the catheter inserted in the right jugular vein.

### Example 3

### Measurement of Pulmonary Arterial Pressure (PAP)

To determine the effect of the chronic hypoxia treatment-induced CH-PAH and to determine the effect of DHEA administration on PAP, the following PAP measurement method was used. Rats were anaesthetized with ketamine 50 mg/kg and xylazine 10 mg/kg by i.p. injection. Mean PAP was measured with 2.5-F catheters inserted into the right jugular vein in closed-chest rats (Bonnet, S., et al. (2002) Cardiovasc. Res. 53, 1019-1028, which is incorporated by reference herein in its entirety). The effect of DHEA on the systemic blood pressure was controlled by another catheter placed into the left carotid artery. Throughout all the experiments, heart rate and oxygen saturation were monitored. Acute hypoxic stress with fraction of inspired O₂ of 10% were applied by administration of an air plus nitrogen gas mixture. The fraction of inspired O₂ was monitored with an oxygen analyzer (Servomex, Crowborough, Great Britain), and the effects on the PAP were measured over a 10-15 minute period.

### Example 4

### Echocardiography Measurements

Echocardiography measurements were also performed to determine differences between the treated and control rats. To perform this procedure, the rats were first anaesthetized. After thorax epilation, the animals were placed in the left lateral decubitus position. Resting echocardiography measurements were performed at ambient conditions by using a SONOS 5500 (Philips) echocardiograph and a 12-MHz sector scare transducer. Right and left ventricle wall thickness was measured on M-mode tracings following the recommendations of the American Society of Echocardiology as described by Jones et al. ((2002) Am. J. Physiol. 283, H364-H371). Right cardiac output was evaluated by measuring the electrical R-R delay, the PA diameter, and the velocity time integral (VTI) of PA flux.

### Example 5

### Exposure to Chronic Hypoxia Results in Increased PAP and Increased RV Wall Thickness

Chronic hypoxic exposure of rats following the method of Example 2 induced PAH, as indicated by a significant increase in PAP from 12.5±0.7 mmHg (N=13) to 18.9±1.7 (N=4), 25.3±1.14 (N=5), and 34.5±1.7 mm Hg (N=13) in control, 1-, 2-, and 3-wk CH rats, respectively (P<0.05) (FIG. 1A). PAHT was also accompanied by an increase in the RV wall thickness from 0.107±0.005 cm (N=10) to 1.91±0.16 mm (N=5), 2.24±0.2 mm (N=7), and 2.74±0.5 mm (N=8) in control, 1-, 2-, and 3-wk CH rats, respectively (P<0.05) (FIG. 1B). The variation in the RV wall thickness was significantly correlated to the increase in PAP (Pearson correlation coefficient=0.84 P<0.02) when determined after 7, 15, and 21 days of CH.

### Example 6

### Effects of DHEA Administration During the CH Treatment

The oral administration of DHEA to normoxic rats had no effect on both PAP and RV wall thickness (FIGS. 1 A and B). However, DHEA administration had a significant effect on rats being given the hypoxia exposure. After 1 or 2 wk of hypoxia exposure with DHEA, no difference was observed in both PAP and RV wall thickness between CH-DHEA and control rats (FIGS. 1 A and B). After 3 wk of hypoxia exposure, the two parameters were significantly decreased in the CH-DHEA group compared with CH groups 21.7±1.1 mm Hg (N=9) vs. 34.5±1.7 mm Hg (N=13), and 1.6±0.03 mm (N=10) vs. 2.74±0.5 mm (N=8) (P<0.05) (FIG. 1B). Therefore, this demonstrates that DHEA administration during the chronic hypoxia treatment has a significant preventive effect on both PAP and RV wall thickness changes. No significant changes in systemic blood pressure, heart rate, and cardiac output were observed in the control, DHEA treated, and untreated groups (FIG. 1C).

### Example 7

### Intravascular Administration of DHEA Partially Reverses Chronic PAH

The above example shows that DHEA administration during the hypoxia treatment can prevent the increase in PAH that CH causes. To determine whether CH-PAH can also be reversed by the addition of DHEA, rats experiencing chronic hypoxia for three weeks were then administered DHEA by either intravascular or oral routes.

Intravascular Administration: Rats were kept in a chronic hypoxic environment for three weeks as described in Example 2. The administration of DHEA at 3 mg/kg as an acute intravascular treatment was delivered within the PA through a catheter. Ten minutes after administration of DHEA (FIG. 2), PAP significantly decreased from 34.5±1.7 to 24.4±2.5 mmHg (N=6) (P<0.05) (FIG. 2B). The intravascular administration of DHEA with successively increasing doses from 30 μg to 3 mg/kg (N=4) resulted in a decrease of the PAP in a dose-dependent manner (FIG. 3).

In contrast, in the 3-week CH-DHEA treated rats, an additional intravascular administration of DHEA did not further reduce PAP (FIG. 2C).

Oral administration: CH rats were kept for three weeks in a chronic hypoxic environment as described in Example 2. During the last week, DHEA was administered orally. This DHEA treatment partially but significantly reversed PAP from 35.9±1.9 mmHg (no DHEA) (N=9) to 21±1.1 mm Hg (+DHEA) (N=3). The DHEA treatment also decreased RV wall thickness from 2.97±0.4 mm (no DHEA) (N=6) to 1.91±0.2 (+DHEA) (N=4), P<0.05. Additionally, these one week DHEA treatments did not affect systemic pressure, cardiac output, or heart rate.

### Example 8

### DHEA Treatment Restores the Pressure Response of the PA to Acute Hypoxia

In addition to the effect of DHEA on rats exposed to chronic hypoxia, the effect of DHEA on the response to an acute hypoxic challenge was determined. Normoxic rats responded to an acute hypoxic challenge by an increase in PAP of 10.1±0.4 mmHg (N=5) after 10-15 minutes. After 3 weeks of hypoxia, however, the pressure response to an acute hypoxic challenge disappeared. In contrast, under DHEA treatment, the pressure response to an acute hypoxic challenge was restored.

### Example 9

### DHEA Treatment Prevents and Reduces CH-Induced IPA Remodeling

CH induces remodeling of the intrapulmonary artery (IPA) in the rat model of CH-PAH. To examine the changes in the IPA wall upon CH exposure, the following method was performed. Isometric contractions were measured in rings from IPAs that were mounted in vertical 5-ml organ baths of a computerized isolated organ bath system (IOX, EMKA Technologies, Paris), as described in Bonnet, S., et al. (2001, Am. J. Physiol. 281, L183-L192, which is incorporated by reference herein in its entirety). Baths were filled with Krebs-Henseleit solution (composition in mM: 118.4 NaCl/4.7 KCl/2.5 CaCl₂/1.2 MgSO₄/1.2 KH₂PO₄/25 NaHCO₃/11.1 D-glucose, pH 7.4). The baths were maintained at 37° C. and bubbled with a 95% O₂/5% CO₂ gas mixture. The tissues were set at optimal length by equilibration against a passive load of 10 mN for rings obtained from normoxic rats and 20 mN for rings obtained from hypoxic rats (Jones, J. E., et al. (2002) Am. J. Physiol. 283, H364-H371, which is incorporated by reference herein in its entirety). A cumulative concentration-response curve was then constructed. A concentration increment was made once the maximal contractile effect of the preceding concentration had been recorded.

Three weeks of CH exposure induced a remodeling of the IPA (<150 μm) wall, which was characterized by an increase of 33% (n=40; N=4 each) in the media layer thickness compared with control rats (FIG. 4). In CH-DHEA rats, the media layer thickness was increased only 11% (n=40; N=4) compared with the control rats, demonstrating that DHEA has a significant protective effect on CH-induced IPA remodeling. Further, the administration of DHEA during the last week, in rats kept 3 weeks in a CH environment, also partially but significantly reversed media wall thickness remodeling (FIG. 4).

### Example 10

### Effect of Chronic Treatment of Rats with DHEA on [Ca²⁺]i of PASMCs

As mentioned earlier, Ca²⁺ is thought to play an important role in the response to chronic hypoxia. To measure the effects of DHEA on [Ca²⁺]i of individual PASMCs, a [Ca²⁺]i-sensitive fluorophore, indo-1 (Calbiochem, San Diego, Calif.), was employed. PASMCs were loaded with indo-1 by incubation in physiological salt solution (139.6 mM NaCl/6.2 mM KCl/1 mM MgCl₂/12.1 mM glucose/10 mM Hepes) containing 1 μM indo-1 penta-acetoxymethyl ester (Indo-1 a.m.) for 25 minutes at room temperature. The recording system included a Nikon Diaphot inverted microscope fitted with epifluorescence. The studied cell was illuminated at 360 nm and counted simultaneously at 405 nm and 480 nm by two photomultipliers (P100, Nikon). The fluorescence ratio (405:480) was calculated on-line and displayed with the two voltage signals on a monitor. [Ca²⁺]i was estimated from the 405:480 ratio (Bonnet, S., et al. (2001) Am. J. Physiol. 281, L193-L201).

Chronic hypoxia induced a significant increase of the resting [Ca²⁺]i value of PASMCs from 69.2±2.2 nM (n=41; N=4) to 143±7 nM (n=51; N=4) in control and 3-wk CH rats, respectively (P<0.05) (FIG. 5). This increase was significantly inhibited in PASMCs from CH-DHEA rats, in which the resting [Ca²⁺]i value was 90.1±3.2 nM (n=30; N=4) (P<0.05). In another group of rats, administration of DHEA from day 15 to day 21 of chronic hypoxia resulted in a significant decrease in the resting [Ca²⁺]i value compared with the untreated rats: 105±3 nM (n=30; N=4) vs. 143±7 nM (n=51; N=4) (P<0.05). This demonstrates that DHEA administration is capable of both partial prevention and partial reversal of CH-induced [Ca²⁺]i increases in PASMCs.

### Example 11

### DHEA Activates BKCa and Kv Channels by a Redox-Sensitive Pathway

To further examine how DHEA functions to decrease the [Ca²⁺]i in PASMCs, the following experiment was performed. PASMCs from rats treated to induce CH as shown in Example 2 were given a 15 minute in vitro treatment with 100 μM DHEA. This treatment significantly decreased the resting [Ca²⁺]i value from 143±7 nM (n=51; N=4) to 82±3 nM, (n=20; N=4) (P<0.05), (FIG. 6A). In contrast, DHEA (100 μM) did not alter [Ca²⁺]i in PASMCs from normoxic, normoxic-DHEA, and CH-DHEA rats (n=30; N=4 per group).

Treatment of PASMCs from CH rats with both DHEA (100 μM) and IbTx (100 nM) for 10 min partially inhibited the change induced by DHEA alone on the resting [Ca²⁺]i value by 62% (n=30; N=4) (FIG. 6A). The addition of agitoxin-2 had no effect (n=15; N=4). Combined presence of 4-AP and IbTx almost totally abolished the DHEA effect on the resting [Ca²⁺]i (n=20; N=4).

Several inhibitors of different signaling pathways were tested. Treatment of PASMCs with DHEA and 1H-[1,2,4]oxadiazolol[4,3,-a]quinoxalin-1-one (ODQ; 10 μM), or genistein (1 μM) or the protein kinase A inhibitor 476485 (1 μM) (n=20; N=4 each) did not alter the effect of DHEA on the resting [Ca²⁺]i value (FIG. 6B). In contrast, DTT, a reducing agent totally inhibited the effect of DHEA (n=30; N=4) and induced a slight elevation of the resting [Ca²⁺]i of 23 nM (FIG. 6B). These results demonstrate that DHEA acts by a redox-sensitive pathway to activate BKCa and Kv channels.

### Example 12

### DHEA Increases BKCa Channel Activity and Expression in PA of CH Rats

Chronic hypoxia had no significant effects on the IbTx induced contraction of IPA rings from CH rats (n=11; N=4) compared with control rats (n=11; N=4) (P>0.05). DHEA administration increased the sensitivity for IbTx in IPA rings (FIG. 7). The tension induced by 100 and 300 nM IbTx was increased by 52% and 55% in rings from CH-DHEA rats compared with control and CH rats.

The level of expression of BKCa was examined using an immunoblot. Arterial pulmonary extracts were prepared from a pool of eight rats per sample and condition. Three separate samples were taken for each condition. The samples were ground in liquid N2, homogenized in RIPA buffer (1% Nonidet P-40/0.5% deoxycholate sodium/0.1% SDS/10 μg/ml aprotinin/100 μg/ml leupeptin/1 mM 4-(2-aminoethyl)benzenesulfonylfluoride in PBS) with a polytron (Bioblock Scientific), and then centrifuged. The extract was verified by Coomassie blue staining. Each sample was loaded at 10 μg in 7.5% resolving acrylamide gel and electrotransferred on to a nylon membrane. Immunoblot analyses used rabbit mAb against rat BKCa (Alomone Labs, Jerusalem, no. APC-021 at 1/500) and mouse monoclonal anti-actin (Santa Cruz Biotechnology, clone C-2 at 1/500). The secondary antibody coupled with peroxidase was used at 1/5,000 dilution (Santa Cruz Biotechnology) and revealed with an enhanced chemiluminescence kit (Amersham Pharmacia). All experiments were repeated two or three times. Quantification of the data was performed using the SCION IMAGE program from the National Institutes of Health.

Immunoblot analysis determined that the BKCa-subunit was recognized by the antibody as a 125-kDa immunoreactive band. The BKCa-subunit was found to be significantly down-regulated in the CH group vs. control group. This decrease was prevented by DHEA orally administered for 3 weeks. No difference was observed in the 45-kDa-actin bands used as an internal standard. Quantitation of the immunoreactive signal associated with the BKCa-subunit was 48.6±5.6% higher in CH-DHEA than CH, (experiment performed in three separate comparisons).

### Example 13

### Treatment of Pulmonary Artery Hypertension in Humans by Inhalation of DHEA

A patient diagnosed with pulmonary artery hypertension is treated with DHEA. The patient self-administers a 1 mg/kg dose of DHEA in the morning and in the evening, administered using a nebulizer. Results are measured weekly. Pulmonary arterial pressure is reduced through this treatment regimen.

### Example 14

Treatment of Pulmonary Artery Hypertension in Humans by Inhalation of DHEA Using a Metered Dose Inhaler

A patient diagnosed with pulmonary artery hypertension is treated with DHEA. The patient self-administers a 0.8 mg/kg dose of DHEA four times per day, and additionally throughout the day as needed, administered using a metered dose inhaler. Results are measured weekly. Pulmonary arterial pressure is reduced through this treatment regimen.

### Example 15

### Treatment of Pulmonary Artery Hypertension in Humans by Inhalation of DHEAS

A patient diagnosed with pulmonary artery hypertension is treated with DHEAS. The patient self-administers a 1.5 mg/kg dose of DHEAS four times per day, administered using a nebulizer at approximately 20 minutes per administration. Results are measured weekly. Pulmonary arterial pressure is reduced through this treatment regimen.

### Example 16

### Treatment of Pulmonary Artery Hypertension in Humans by Injection of DHEA

A patient diagnosed with pulmonary artery hypertension is treated with an injection of sterile DHEA at a pH of 7.0, twice a week. The amount of DHEA administered is 3.0 mg/kg of patient body weight. Results are measured weekly. Pulmonary arterial pressure is reduced through this treatment regimen.

### Example 17

### Treatment of Pulmonary Artery Hypertension in Humans by Oral Administration of DHEA

A patient diagnosed with pulmonary artery hypertension is treated three times per day with an oral dose of DHEA. The patient self-administers a capsule containing 1 mg/kg of DHEA three times per day. Results are measured weekly. Pulmonary arterial pressure is reduced through this treatment regimen.

### Example 18

### Reversal of Pulmonary Artery Hypertension in Humans by Pulmonary Administration of DHEA

A patient diagnosed with pulmonary artery hypertension is administered DHEA at 2.0 mg/kg of patient body weight, on a daily dosage basis, using a metered dose inhaler. Results are measured weekly. Pulmonary arterial pressure is reduced through this treatment regimen.

### Example 19

### Prevention of Pulmonary Artery Hypertension in Humans by Administering DHEA by an Oral Route

A patient at risk for developing pulmonary artery hypertension is administered an oral dose of DHEA at 3.0 mg/kg of patient body weight, two times per week. The onset of increased pulmonary arterial pressure and pulmonary hypertension is delayed or prevented using this regimen.

### Example 20

### Prevention of Pulmonary Artery Hypertension in Humans by Pulmonary Administration of DHEA

A patient at risk for developing pulmonary artery hypertension is administered a dose of 2.0 mg/kg DHEA, approximately two times per week, using a metered dose inhaler. The onset of increased pulmonary arterial pressure and pulmonary hypertension is delayed or prevented using this regimen.

Pulmonary artery hypertension may be present in addition to other diseases. For example, microbial infections may co-exist with pulmonary artery hypertension, exacerbating the health problems of the individual. Examples 21 through 24 provide illustrations of methods of treating such infections and pulmonary artery hypertension by treating with a combination of DHEA plus an antimicrobial agent.

### Example 21

### Treatment of Pulmonary Artery Hypertension in Combination with a Pulmonary Bacterial Infection with a Combination Treatment of DHEA Plus an Antibacterial Agent Using a Metered Dose Inhaler

A patient diagnosed with pulmonary artery hypertension is treated with DHEA. The patient self-administers a pharmaceutical composition comprising 1.2 mg/kg dose of DHEA plus 30 mg of the antibiotic amoxicillin four times per day, administered using a metered dose inhaler. Results are measured weekly. Pulmonary infection and pulmonary arterial pressure are reduced through this treatment regimen.

### Example 22

### Treatment of Pulmonary Artery Hypertension in Combination with a Pulmonary Bacterial Infection by a Combination Treatment of DHEA Administered Using a Nebulizer and an Oral Dose of an Antibacterial Agent

A patient with pulmonary artery hypertension and the presence of a pulmonary bacterial infection is treated 3 times per day with a 1.0 mg/kg dose of DHEA using a nebulizer. Additionally, a 300 mg oral capsule dose of the antibiotic erythromycin is administered once per day. The level of bacterial infection in the lungs, along with pulmonary arterial pressure, are determined twice a week. Both pulmonary arterial pressure and pulmonary bacterial infection are reduced through this treatment regimen.

### Example 23

### Treatment of Pulmonary Artery Hypertension in Combination with a Pulmonary Fungal Infection by a Combination Treatment of DHEA Plus an Antifungal Agent Using a Metered Dose Inhaler

A patient diagnosed with both pulmonary artery hypertension and a pulmonary fungal infection is treated 5× per day with a pharmaceutical composition containing a combination of DHEA (at 0.8 mg/kg per administration) plus 10 mg of the antifungal agent fluconazole (Diflucan) using a metered dose inhaler. Fungal infection and pulmonary arterial pressure are measured weekly. Pulmonary arterial pressure and the pulmonary fungal infection are reduced through this treatment regimen.

### Example 24

### Treatment of Pulmonary Artery Hypertension in Combination with a Pulmonary Viral Infection by a Combination Treatment of DHEAS Plus an Anti-Viral Agent

A patient exhibiting symptoms of a pulmonary viral infection along with pulmonary artery hypertension is treated with a combination of DHEAS and the anti-viral agent acyclovir. The patient self-administers a pharmaceutical composition comprising 1.2 mg/kg dose of DHEAS plus 40 mg of the anti-viral agent acyclovir twice daily, using a nebulizer for 20 minutes per administration. Results are measured weekly. Pulmonary arterial pressure and pulmonary viral infection are reduced through this treatment regimen.

DHEA, DHEAS, DHEA analogs, or DHEA derivatives may also be useful when administered in combination with vasodilators or bronchodilators for the treatment of numerous pulmonary diseases. The following examples 25 and 26 illustrate methods that may be used to treat a patient diagnosed with a pulmonary disease, using a combination treatment method.

### Example 25

### Reduction of the Severity of Pulmonary Diseases with a Combination of the Pulmonary Administration of DHEA Plus Oral Administration of a Vasodilator

A patient diagnosed with a pulmonary disease is treated with a combination of DHEA and a vasodilator. The patient self-administers a pharmaceutical composition comprising 0.7 mg/kg dose of DHEA using a metered dose inhaler. Additionally, an oral dose of 50 mg of the vasodilator nifedipine is administered once per day. Results are measured weekly. The severity of the pulmonary disease is reduced, and pulmonary arterial pressure is reduced through this treatment regimen.

### Example 26

### Treatment of Pulmonary Diseases with a Combination of DHEA Plus a Bronchodilator

A patient diagnosed with a pulmonary disease is treated three times per day with a combination of 1.0 mg/kg dose of DHEA and a 100 μg dose of the bronchodilator albuterol, using a nebulizer. Results are measured twice a week. The pulmonary disease is diminished and pulmonary arterial pressure is reduced through this treatment regimen.

It will be appreciated that no matter how detailed the foregoing appears in text, the invention can be practiced in many ways. Thus, although this invention has been described in terms of certain preferred embodiments, other embodiments which will be apparent to those of ordinary skill in the art in view of the disclosure herein are also within the scope of this invention. Accordingly, the scope of the invention is intended to be defined only by reference to the appended claims and any equivalents thereof. All documents cited herein are incorporated herein by reference in their entireties.

1. A method for reducing pulmonary arterial pressure (PAP), comprising introducing an effective amount of DHEA, DHEAS, DHEA analog, or DHEA derivative into the pulmonary airways of a mammal.

2. The method of claim 1, wherein said DHEA, DHEAS, DHEA analog, or DHEA derivative has the general formula: 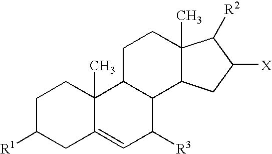
wherein X is H or halogen; R¹ R² and R³ are independently ═O, —OH, —SH, H, halogen, a pharmaceutically acceptable ester, a pharmaceutically acceptable thioester, a pharmaceutically acceptable ether, a pharmaceutically acceptable thioether, a pharmaceutically acceptable inorganic ester spirooxirane, spirothirane, —OSO₂R⁵, —OPOR⁵R⁶, or a pharmaceutically acceptable monosaccharide, disaccharide or oligosaccharide; wherein R⁵ and R⁶ are independently —OH, a pharmaceutically acceptable ester, a pharmaceutically acceptable ether; or a pharmaceutically acceptable salt, and alternatively wherein R² and R³ are selected from the group consisting of a methyl group, a partially or completely dehydrogenated aliphatic hydrocarbon chain of 2-14 carbons, and a saturated aliphatic hydrocarbon chain of 2-14 carbons.

3. The method of claim 2, wherein R¹, R², R³, and X are selected from the group consisting of:
R² is ═O, R³ and X are each H and R¹ is ═O, —OH, a pharmaceutically acceptable esters ester thereof, pharmaceutically acceptable ethers thereof or pharmaceutically acceptable salts;
R² is ═O, R³ is H, X is halogen and R¹ is ═O, —OH, pharmaceutically acceptable esters thereof, pharmaceutically acceptable ethers thereof or pharmaceutically acceptable salts;
R² is ═O, R³ and X are each H and R¹ is —SH, pharmaceutically acceptable thioesters thereof, pharmaceutically acceptable thioethers thereof or pharmaceutically acceptable salts;
R² is ═O, R³ is H. X is halogen and R¹ is —SH, pharmaceutically acceptable thioesters thereof, pharmaceutically acceptable thioethers thereof or pharmaceutically acceptable salts;
R² is ═O, X is H and R¹ and R³ are independently ═O, —OH, pharmaceutically acceptable esters thereof, pharmaceutically acceptable ethers thereof or pharmaceutically acceptable salts;
R² is ═O, X is halogen and R¹ and R³ are independently ═O, —OH, pharmaceutically acceptable esters thereof, pharmaceutically acceptable ethers thereof or pharmaceutically acceptable salts;
R² is ═O, X is H and R¹ and R³ are independently —SH, pharmaceutically acceptable thioesters thereof, pharmaceutically acceptable thioethers thereof or pharmaceutically acceptable salts;
R² is ═O, X is halogen and R¹ and R3 are independently —SH, pharmaceutically acceptable thioesters thereof, pharmaceutically acceptable thioethers thereof or pharmaceutically acceptable salts;
R² is —OH, R³ and X are each H and R¹ is ═O —OH, pharmaceutically acceptable esters thereof, pharmaceutically acceptable ethers thereof or pharmaceutically acceptable salts; R² is —OH, R³ is H, X is halogen and R¹ is ═O —OH, pharmaceutically acceptable esters thereof, pharmaceutically acceptable ethers thereof or pharmaceutically acceptable salts; R² is —OH, R³ and X are each H and R¹ is —SH, pharmaceutically acceptable thioesters thereof, pharmaceutically acceptable salts;
R² is —OH, R³ is H, X is halogen and R¹ is —SH, pharmaceutically acceptable thioesters thereof, pharmaceutically acceptable thioethers thereof or pharmaceutically acceptable salts;
R² is —OH, X is H and R¹ and R³ are independently ═O, —OH, pharmaceutically acceptable esters thereof, pharmaceutically acceptable ethers thereof or pharmaceutically acceptable salts;
R² is —OH, X is halogen and R¹ and R³ are independently ═O, —OH, pharmaceutically acceptable esters thereof, pharmaceutically acceptable ethers thereof or pharmaceutically acceptable salts;
R² is —OH, X is H and R¹ and R³ are independently —SH, pharmaceutically acceptable thioesters thereof, pharmaceutically acceptable thioethers thereof or pharmaceutically acceptable salts;
R² is —OH, X is halogen and R¹ and R³ are independently —SH, pharmaceutically acceptable thioesters thereof, pharmaceutically acceptable thioethers thereof or pharmaceutically acceptable salts;
R² is —SH, R³ and X are each H and R¹ is ═O —OH, pharmaceutically acceptable esters thereof, pharmaceutically acceptable ethers thereof or pharmaceutically acceptable salts;
R² is —SH, R³ is H, X is halogen and R¹ is ═O —OH, pharmaceutically acceptable esters thereof, pharmaceutically acceptable ethers thereof or pharmaceutically acceptable salts;
R² is —SH, R³ and X are each H and R¹ is —SH, pharmaceutically acceptable thioesters thereof, pharmaceutically acceptable thioethers thereof or pharmaceutically acceptable salts
R² is —SH, R³ is H, X is halogen and R¹ is —SH, pharmaceutically acceptable thioesters thereof, pharmaceutically acceptable thioethers thereof or pharmaceutically acceptable salts;
R² is —SH, X is H and R¹ and R³ are independently ═O, —OH, pharmaceutically acceptable esters thereof, pharmaceutically acceptable ethers thereof or pharmaceutically acceptable salts;
R² is —SH, X is halogen and R¹ and R³ are independently ═O, —OH, pharmaceutically acceptable esters thereof, pharmaceutically acceptable ethers thereof or pharmaceutically acceptable salts;
R² is —SH, X is H and R¹ and R³ are independently —SH, pharmaceutically acceptable thioesters thereof, pharmaceutically acceptable thioethers thereof or pharmaceutically acceptable salts;
R² is —SH, X is halogen and R¹ and R³ are independently —SH, pharmaceutically acceptable thioesters thereof, pharmaceutically acceptable thioethers thereof or pharmaceutically acceptable salts;
X is H and R¹, R² and R³ are independently ═O, —OH, a sugar residue, pharmaceutically acceptable esters thereof, pharmaceutically acceptable ethers thereof or pharmaceutically acceptable salts, wherein at least one of R¹, R² and R³ is a sugar residue;
X is halogen and R¹, R² and R³ are independently ═O, —OH, a sugar residue, pharmaceutically acceptable esters thereof, pharmaceutically acceptable ethers thereof or pharmaceutically acceptable salts, wherein at least one of R¹, R² and R³ is a sugar residue;
X is H and R¹, R² and R³ are independently ═O, —OH, pharmaceutically acceptable inorganic esters thereof or pharmaceutically acceptable salts, wherein at least one of R¹, R² and R³ is an inorganic ester;
X is halogen and R¹, R² and R³ are independently ═O, —OH, pharmaceutically acceptable inorganic esters thereof or pharmaceutically acceptable salts, wherein at least one of R¹, R² and R³ is an inorganic ester.

4. The method of claim 1, wherein said mammal is a human.

5. The method of claim 1, wherein said introducing is by inhalation, pulmonary administration, inspiration, aerosolization or nebulization.

6-9. (canceled)

10. The method of claim 1, wherein said effective amount of DHEA, DHEAS, DHEA analog, or DHEA derivative is from about 0.01 mg per kg body weight to about 100 mg per kg body weight per day.

11. The method of claim 1, wherein said introducing an effective amount of DHEA, DHEAS, DHEA analog, or DHEA derivative is by chronic administration.

12. The method of claim 1, wherein said introducing an effective amount of DHEA is by intermittent administration.

13. The method of claim 1, wherein the DHEA, DHEAS, DHEA analog, or DHEA derivative is in the form of a dry particulate.

14. The method of claim 1, wherein the DHEA, DHEAS, DHEA analog, or DHEA derivative is in the form of an aerosol.

15. The method of claim 14, wherein said effective amount of DHEA, DHEAS, DHEA analog, or DHEA derivative is from about 0.01 mg per kg body weight to about 100 mg per kg body weight per day.

16. The method of claim 1, further comprising administration of an antibacterial agent, antifungal agent, antiviral agent, vasodilator, bronchodilator or anti-inflammatory agent.

17-21. (canceled)

22. A metered dose inhaler comprising at least one compound selected from the group consisting of: DHEA, DHEAS, a DHEA analog, and a DHEA derivative.

23. The metered dose inhaler of claim 22, further comprising an antibacterial agent, antifungal agent, antiviral agent, bronchodilator, vasodilator or anti-inflammatory agent.

24-29. (canceled)

30. A dry powder inhaler comprising a formulation comprising at least one compound selected from the group consisting of: DHEA, DHEAS, a DHEA analog, and a DHEA derivative.

31. The inhaler of claim 30, wherein the DHEA formulation has a particle size of about 0.5 μm to about 5 μm.

32. A method of treating pulmonary artery hypertension in an individual, comprising administration of an effective amount of a composition comprising at least one compound selected from the group consisting of: DHEA, DHEAS, a DHEA analog, and a DHEA derivative.

33. The method of claim 32, wherein said individual is a mammal.

34. The method of claim 32, wherein said administration is oral, pulmonary or by injection.

35-36. (canceled)

37. The method of claim 34, wherein said pulmonary administration comprises an aerosol.

38. The method of claim 32, wherein said pulmonary artery hypertension is caused by disorders of the respiratory system.

39. The method of claim 32, wherein said pulmonary artery hypertension is caused by chronic hypoxia.

40. The method of claim 39, wherein said pulmonary artery hypertension is chronic hypoxic pulmonary artery hypertension.

41. The method of claim 33, wherein said mammal is a human.

42. A method of reversing the severity of pulmonary artery hypertension in an individual, comprising administering an effective amount of at least one compound selected from the group consisting of: DHEA, DHEAS, a DHEA analog, and a DHEA derivative.

43. A method of decreasing RV wall thickness in a mammal, comprising administering an effective amount of a composition comprising at least one compound selected from the group consisting of: DHEA, DHEAS, a DHEA analog, and a DHEA derivative.
